(19) United States
(12) Patent Application Publication
(10) Pub. No.: US 2003/0065334 A1
(43) Pub. Date: Apr. 3, 2003

(54) Cast removal device

(76) Inventors: Randy R. Hobgood, Henderson, KY (US); Sheryl L. Shelton, Henderson, KY (US)

Correspondence Address: C RICHARD MARTIN, MARTIN & MARTIN, PO BOX 29, BOONVILLE, IN, 47601, US

(21) Appl. No.: 09/968,635

(22) Filed: Sep. 29, 2001

(51) Int. Cl. A61F 5/04

(52) U.S. Cl. 606/105.5

(57) ABSTRACT

A cast removal device having a blade assembly driven by a cordless tool is provided. Mirror image opposing first and second blade members having cutting edges along a lower inner edge thereof are disposed in parallel with a third blade member sandwiched in between. Each of the first and second blade members has a first planar surface and a second planar surface, a first longitudinal edge and a second longitudinal edge, first and second ends, and an opening disposed proximal to a midpoint. The third blade member has a first planar surface and a second planar surface, a first longitudinal edge and a second longitudinal edge, first and second ends, and a pivot point disposed proximal to a midpoint thereof. Means for rotatably connecting the first, second and third blade members at the pivot points thereof are provided as well as means for securing the first and second blade members together to prevent rotation with respect to one another. Drive means are also provided for effecting oscillation of the third blade member in relation to the first and second blade members about axis of rotation to permit a sort of double-scissor action upon actuation of the device.

### BACKGROUND OF INVENTION

The present invention relates to devices for removal of casts used to set broken bones to enable them to heal properly, and more particularly relates to an improved device for removal of casts quickly and safely without danger of cutting or burning the patient.

Casts normally comprise three layers: a hard outer shell (usually made of fiberglass or plaster) a soft, cushy layer of cotton padding and an inner gauze or stockinet of cotton, polyester or other woven material to be placed against the skin. The purpose of the hard outer shell is to immobilize and protect the body part being cast. The cotton padding and inner stockinet provide a comfortable interface between the outer shell and the patient's skin.

The vast majority of cast removal is accomplished by use of an electric oscillating cast cutter which effects cutting of the cast material by means of a circular oscillating blade. To cut the cast, the operator grasps the tool and engages the rotating blade with the outer shell of the cast. As pressure is put on the blade, it cuts down through the cast. Enough pressure must be applied to cut through the outer shell of the cast. However, the operator must use caution not to apply too much pressure to the blade which would result in cutting through the padding and into the patient's skin. The cast removal is a time consuming and tedious task involving application of pressure to effect cutting, withdrawal of the blade, advancement of the blade along the cutting line, and repeated application of pressure to effect further cutting. Once a cut has been made along the entire length of the cast, a separator must be inserted to create an opening between the newly cut edges. Scissors may then be used to cut the padding and stockinet and the cast may then be removed from the patient.

The use of such a cast cutting saw is particularly hazardous in the removal of so-called direct of total contact casts, where a cast is applied with little or no padding. The likelihood of cutting or burning a patient's skin when removing such a cast has proven to be significant. In addition, use of such an oscillating cast saw creates significant amounts of dust and particulate as the saw blade in effect pulverizes a narrow sliver of the cast to effect its cutting action.

In light of the foregoing, it is a purpose of the present invention to provide a means for effectively and quickly removing a cast from a patient while protecting the patient's skin from cutting or burning. It is a further purpose of the present invention to provide a means for quickly removing a cast from a patient without creating dust or debris. These and other purposes of the present invention will become evident upon review of the following specification.

### SUMMARY OF INVENTION

According to one aspect of the present invention, there is provided a cast removal device comprising first and second blade members and a third blade member. The blade members are preferably formed from steel coated with a layer of ceramic-metal matrix material. Each of the first and second blade members include a first outer planar surface and a second inner planar surface, a first longitudinal edge and a second longitudinal edge, first and second ends, and a pivot point disposed proximal to a midpoint along said first and second planar surfaces, said first and second blade members being positioned parallel to one another such that the second inner planar surface of the second blade member faces the second inner planar surface of the first blade member. The third blade member includes a first planar surface and a second planar surface, a first longitudinal edge and a second longitudinal edge, first and second ends, and a pivot point disposed proximal to a midpoint along said first and second planar surfaces for permitting oscillation about an axis perpendicular to said first and second planar surfaces. The third blade member is positioned between said first and second blade members such that said first planar surface of said third blade member slidably engages said second surface of said first blade member and said second planar surface of said third blade member slidably engages said second planar surface of said second blade member. The first ends of said first, second and third blade members are rounded.

A blade housing is also provided wherein said first and second blade members are fixedly secured to said housing. The blade housing is preferably formed of aluminum. Means for rotatably connecting said first, second and third blade members at the pivot points thereof are also provided. A stepped bushing is provided having a central section and outer sections with the diameter of the center section being greater than the diameter of the outer sections. The diameter of the center section is sized to be received in the central opening in the third blade member and the outer sections are sized to be received in the central openings of the first and second blade members. A fastener passes through the housing, blades and bushing to secure the blades to the housing so that oscillation of the third blade about that axis is permitted. Means for securing said first and second blade members together to prevent rotation with respect to one another are also provided. A second bushing is provided proximal to the second ends of the first and second blades between said blades. A fastener passes through the housing, first blade, bushing and second blade to further secure the first and second blade members to the housing and to prevent rotation of the first and second blade members in relation to the housing.

The third blade preferably includes a V-shaped groove along a portion of the length of the first longitudinal edge such that a first cutting edge is formed at the intersection of the first planar surface and the first longitudinal edge and a second cutting edge is formed at the intersection of the second planar surface and the first longitudinal edge. The walls of the V-shaped groove are inclined at an angle of between 20° and 45°, and preferably 45°, in relation to a plane parallel to the first longitudinal edge. A portion of the length of the first longitudinal edge carrying said first and second cutting edges is curved such that the radius of curvature is between 3″ and 3.5″, and optimally the radius of curvature is 3.2″.

The first and second blade members each include a beveled section along a portion of the length of the first longitudinal edge thereof such that cutting edges are formed at the intersection of the second planar surface and the second longitudinal edge of the first blade member and at the intersection of the second planar surface and the second longitudinal edge of the second blade member. The beveled surfaces are at an angle of between 10° and 20°, and optimally at an angle of 15°, in relation to a plane parallel to the second longitudinal edge.

Drive means for effecting oscillation of the third blade member in relation to the first and second blade members about said axis of rotation. The drive means preferably is an electric drive motor for imparting rotary motion to a shaft passing through windings of said motor. A cylindrical race member is provided with a plurality of needle bearings disposed along the inner surface thereof. A nut, having a stepped down cylindrical race section thereon is provided such that the outer surface of the race section engages the inner surfaces of the needle bearings. The nut has a threaded opening along the length thereof, with said opening being positioned through said race portion of said nut offset from the center point thereof. The second end of said third blade member comprises a semicircular opening therein having cam surfaces for selectively engaging and disengaging the outer surface of the first race member during rotation thereof.

A cover that is removably disposed over the first end of said third blade member may also be provided. The cover is preferably formed of plastic material and comprises a projection proximal to a distal end thereof. A corresponding notch is provided in the second longitudinal edge of the third blade member for receiving said projection therein.

These and other features, aspects and advantages of the present invention will become better understood by referring to the following detailed description, drawings and claims, wherein examples of the presently preferred embodiments are given for purposes of illustration and disclosure.

### BRIEF DESCRIPTION OF DRAWINGS

FIG. 1 is a side perspective view of the blade assembly of the cast removal device according to one preferred embodiment of the invention.

FIG. 2 is a side plan view of the blade assembly shown in FIG. 1.

FIG. 3 is a top plan view of the blade assembly shown in FIG. 1.

FIG. 4 is a reversed perspective view of the second blade member of the blade assembly shown in FIG. 1.

FIG. 5 is a bottom plan view of the second blade member shown in FIG. 4.

FIG. 6 is a side perspective view of the third blade member of the blade assembly shown in FIG. 1.

FIG. 7 is a side view of the blade assembly and blade housing of the cast removal device according to one preferred embodiment of the invention.

FIG. 8 is a cross sectional view of the blade assembly and blade housing shown in FIG. 7 along the line 8-8.

FIG. 9 is a cross sectional view of the blade assembly and blade housing shown in FIG. 7 along the line 9-9.

FIG. 10 is a cross sectional view of the blade assembly shown in FIG. 3 along the line 10-10.

FIG. 11 is a magnified view of the blade assembly shown in FIG. 10 along the line 11-11.

FIG. 12 is a perspective view of a protective cover for the third blade of the blade assembly of the cast removal device according to one preferred embodiment of the invention.

FIG. 13 is a side perspective view of the blade housing of the cast removal device according to one preferred embodiment of the invention.

FIG. 14 is an exploded view of the cast removal device according to one preferred embodiment of the invention.

FIG. 15 is an exploded view of a portion of the drive assembly of the cast removal device according to one preferred embodiment of the invention.

### DETAILED DESCRIPTION

The present invention is best understood by referring to the Drawings in connection with review of this Description. The present invention is a device for quickly and easily removing a multi-layered cast from a patient in a single-step operation.

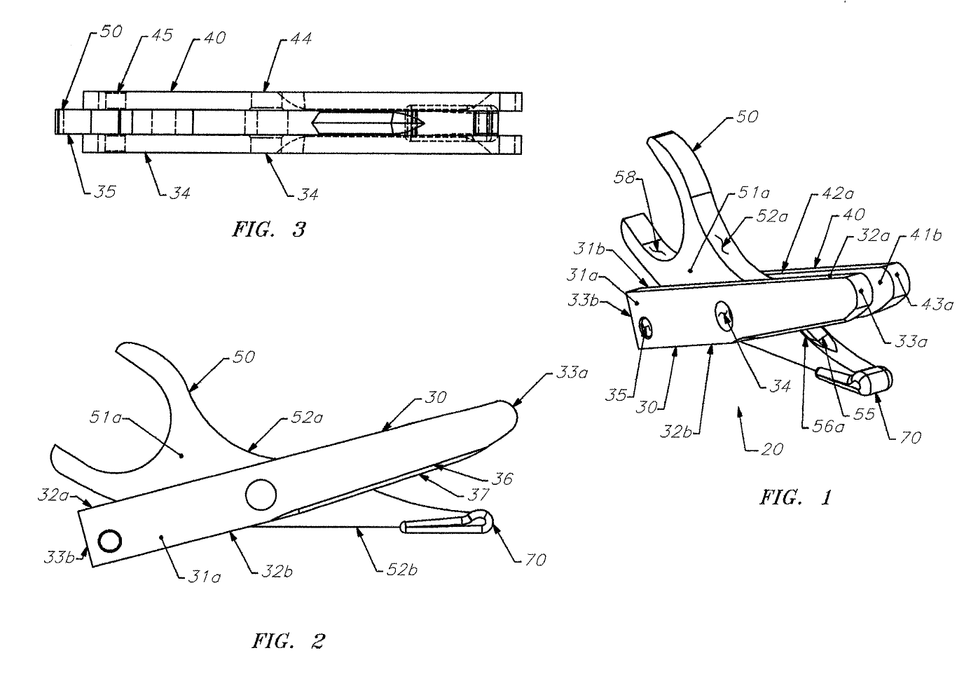

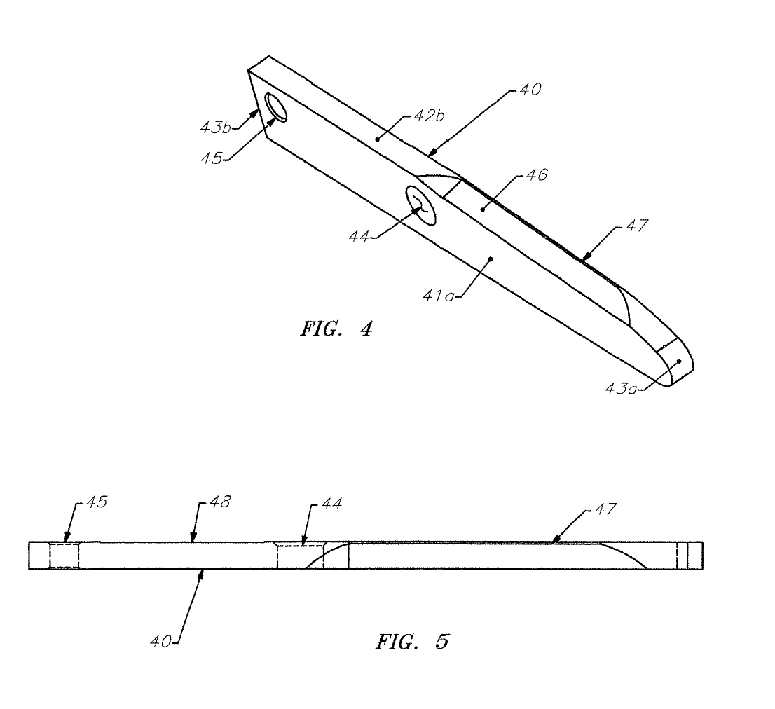

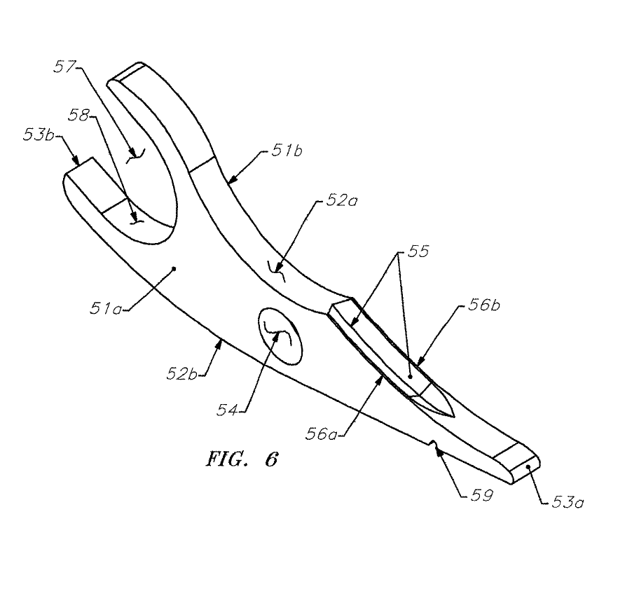

FIGS. 1-6 show the blade assembly 20 of the cast removal device according to a preferred embodiment of the present invention. The blade assembly generally comprises a first blade member 30, second blade member 40, and third blade member 50. The first blade member 30 and second blade member 40 are essentially mirror images of one another. For purposes of convenience, the details of both the first and second blade members will be described in relation to the first blade member. Reference numerals used to designate elements of the first blade member correspond to reference numeral used to designate elements of the second blade member. For example, the outer or first planar surface of the first blade member is designated by reference numeral 31a, while the outer or first planar surface of the second blade member is designated by reference numeral 41a.

The first blade member 30 includes an outer or first planar surface 31a, an inner or second planar surface 31b, an upper or first longitudinal edge 32a, a lower or second longitudinal edge 32b, a first or leading end 33a, and a second or trailing end 33b. An opening 34 is provided near a midpoint of the blade member and passing through the first and second planar surfaces 31a, 31b. The first or leading end 33a is rounded for safety to protect the patient from sharp edges. A second opening 35 is provided for receiving a fastener, such as a bolt, there through to secure the first and second blades to the housing to prevent rotation of said first and second blades in relation to the housing. The second or lower longitudinal edge 32b of the first blade member 30 further includes a beveled section 36 along a portion of the length thereof such that a cutting edge 37 is formed at the intersection of the inner or second planar surface 31b and the second or lower longitudinal edge 32b of the blade member. The beveled surface is preferably at an angle of between 10° and 20° in relation to a plane parallel to the second longitudinal edge, and optimally at an angle of 15° in relation to a plane parallel to the second longitudinal edge. A grease reservoir 38 is formed in the inner surface 33b of the blade member 30. The reservoir 38 is preferably about 0.005″ deep and holds lubricant therein, and, upon heating up of the blades due to friction in operation, lubricant is drawn from the reservoir to lubricate the stepped bearing 60.

The third blade member 50 includes a first planar surface 51a and a second planar surface 51b, a first longitudinal edge 52a and a second longitudinal edge 52b, a first or leading end 53a and a second end 53b. An opening or pivot point 54 is formed through the blade member 50 from the first planar surface to the second planar surface. The opening is located near the midpoint of the blade member for permitting oscillation about an axis perpendicular to said first and second planar surface.

A V-shaped groove 55 extends along a portion of the length of the first longitudinal edge 52a of the third blade member 50 such that a first cutting edge 56a is formed at the intersection of the first planar surface 51a and the first longitudinal edge 52a and a second cutting edge 56b is formed at the intersection of the second planar surface 51b and the first longitudinal edge 52a. The groove 55 and corresponding cutting edges allow the third blade member to quickly and easily cut through the cotton padding and stockinet layers of the cast. The walls of the V-shaped groove are at an angle of between 20° and 45°, and preferably between 30° and 45°, in relation to a plane parallel to the first longitudinal edge. The optimal angle of the walls of the V-shaped groove in relation to a plane parallel to the first longitudinal edge is 45° thus providing the necessary sharpness of the cutting edges while still maintaining strength in the sidewalls thereof. The portion of the length of the first longitudinal edge which carries the first and second cutting edges is curved. The curvature facilitates movement of the cut cast material toward the outside of the housing. The radius of curvature is preferably between 3″ and 3.5″, and optimally 3.2″. The second end 53b of said third blade member includes a semicircular opening 57 therein having cam surfaces 58 thereon.

The first blade member 30 and second blade member 40 are positioned parallel to one another such that the second or inner planar surface 31b of the first blade member 30 faces the second or inner planar surface 41b of the second blade member 40. The third blade member 50 is positioned between said first and second blade members such that the first planar surface 51a of the third blade member slidably engages said second surface 31b of said first blade member and said second planar surface 51b of said third blade member slidably engages said second surface 41b of said second blade member. A stepped bearing 60 (FIG. 8) is provided for rotatably coupling the first and second blade members to the third blade member. The large central section of the stepped bearing 60 is positioned within the central opening 54 of the third blade member 50, while the outer, stepped down portions of the bearing 60 are received within the openings 34, 44 of the first and second blade members 30, 40, respectively. When assembled, the leading ends 33a, 43a of the first and second blade members 30, 40 extend further forward than the leading end 53a of the third blade member 50. This results in a shorter stroke of the pivoting third blade member 50, thereby reducing the likelihood that the third blade member will come into contact with and potentially cut or bum the patient's skin. The blade members are preferably formed from air hardened A2 steel having a Rockwell hardness of approximately 58 or 59 coated with a layer of ceramic-metal matrix material giving the outer surface of the blade a Rockwell hardness of approximately 75. Alternatively, the blade members may be formed of stainless steel.

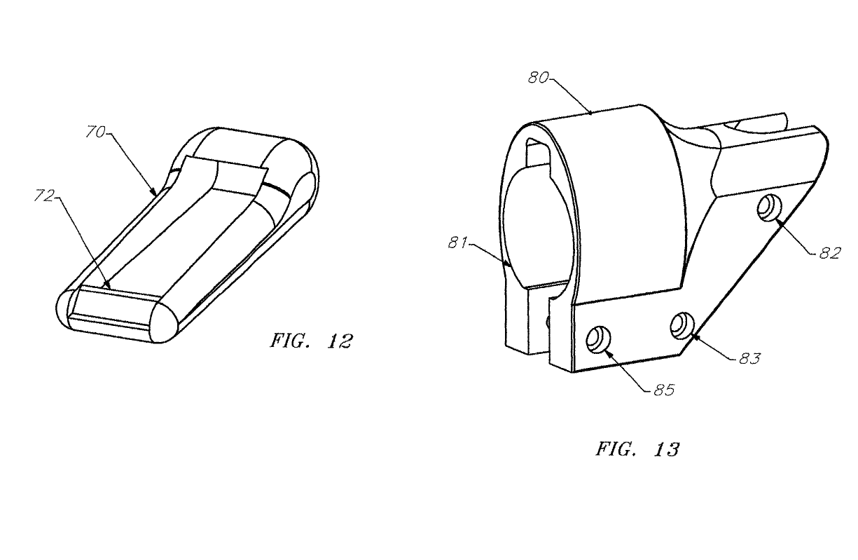

The second longitudinal edge 52b of the third blade member 50 further includes a notch 59 formed in the surface thereof near the leading end 53a. A protective cover 70 may be placed over the leading end 53a of the third blade member to prevent direct contact between the third blade member and the patient's skin. The cover 70 is preferably formed of a plastic or other material with superior heat dissipation characteristics to further reduce burning of the patient's skin which may be caused by heating of the blade due to friction caused by operation of the device. As best shown in FIGS. 11 and 12, the cover further includes a projection 72 on the inner surface thereof for releasably engaging said notch 59 to secure the cover 70 in place.

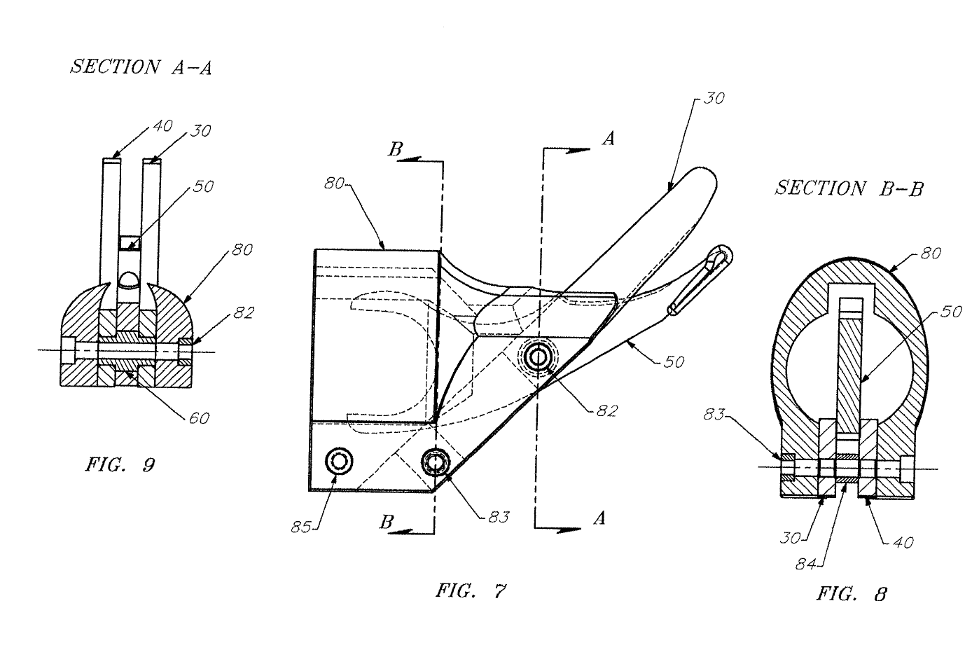

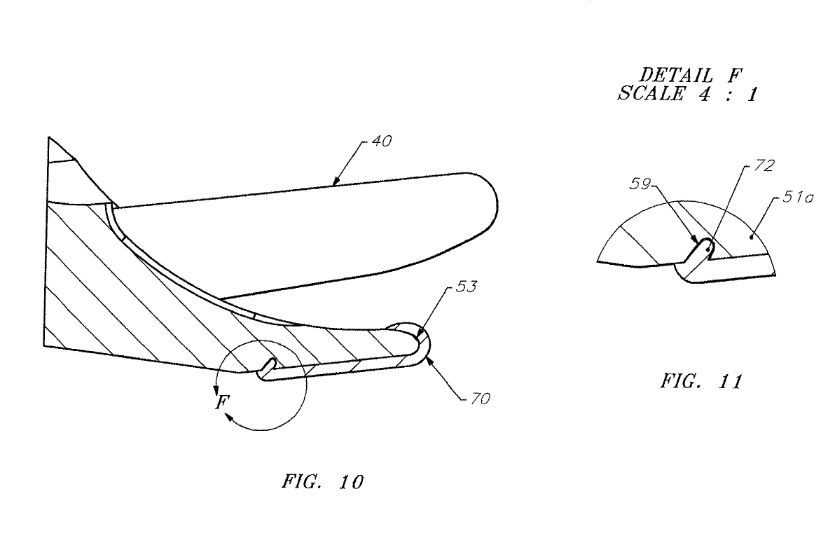

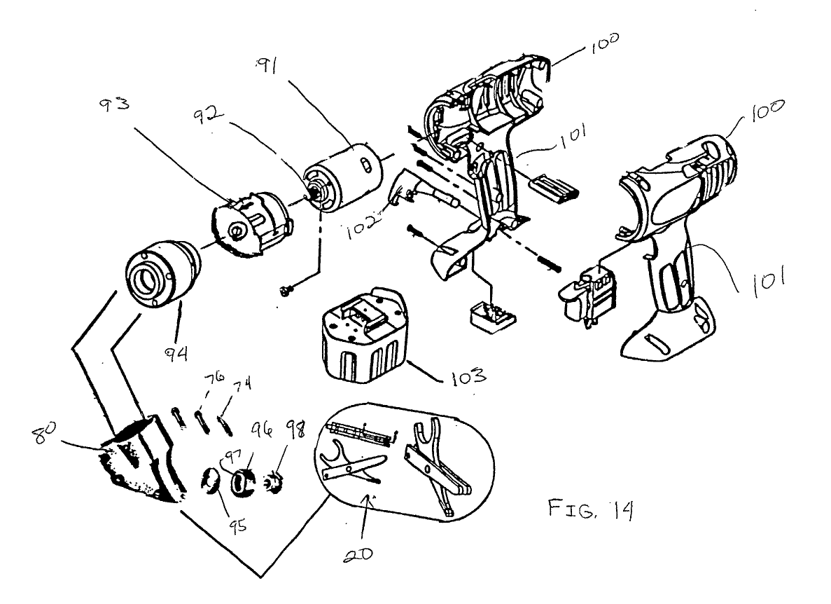

As shown in FIGS. 7-9, 13 and 14, the cast removal device 10 further includes a blade housing 80. The blade housing 80 surrounds and encloses the second or trailing ends 33b, 43b of the first second and third blade members. An opening 81 is provided in a first end of the housing 80, with said opening being of a size and shape to permit coupling of it with the respective drive means 90. According to the preferred embodiment shown in the drawings, the opening 81 is circular in shape to permit coupling with the circular profile of the hub 94 of the drive means 90. The first and second blade members are fixedly secured to the blade housing by a pair of fasteners 74, 76. Preferably each fastener comprises a bolt and corresponding nut or a bolt and corresponding threads formed in an opening in the housing. The first fastener 74 passes through a first opening 82 in one wall of the housing 80, through the stepped bearing 60 and into a second opposing housing wall where it is secured by a threaded connection. The second fastener 76 passes through a second opening 83 in a first wall of the housing, through opening 35 in the first blade member 30, through a bushing 84 positioned between the first and second blade members, through opening 45 in the second blade member 40 and into a second housing wall where it is secured. A third fastener 76 passes through a third opening 85 in the housing and serves to secure the housing 80 to the hub 94. The blade housing preferably is formed of aluminum or another material that dissipates heat well.

The drive means 90 of the cast removal device may take many forms. According to the preferred embodiment of the present invention, the drive means comprises an electric drive motor 91 for imparting rotary motion to a shaft 92 via transmission 93. The shaft 92 then passes through a hub 94 with an end portion thereof protruding therefrom for receiving the race assembly. The race assembly comprises a washer-like member 95, a cylindrical race member 96 with a plurality of needle bearings 97 disposed along the inner surface thereof, and a nut 98. The nut 98 includes a stepped down cylindrical race section 98a thereon such that the outer surface of the race section engages the inner surfaces of the needle bearings 97. The nut 98 has a threaded opening along the length thereof, with said opening being positioned through said race portion 98a of said nut offset from the center point thereof.

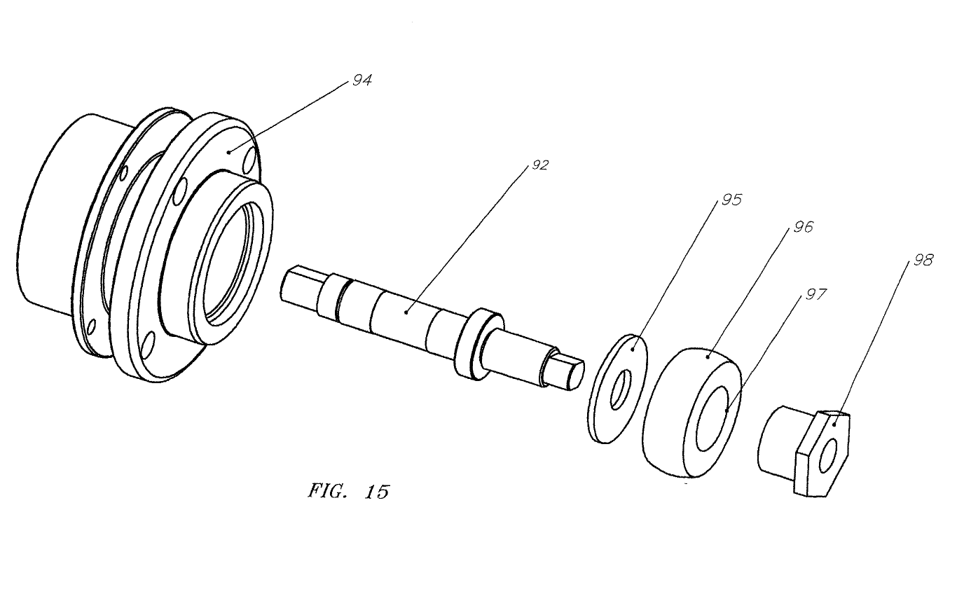

As shown in FIG. 14, the preferred configuration of the drive means 90 is that of a cordless powertool having a housing 100 made of light-weight high-impact plastic, having a handle portion 101 thereof including a trigger 102 for activating the electric motor 91. The motor is preferably powered by a rechargeable battery 103, such as a 12 volt or 14.4 volt NiCad battery. The transmission 93 may helps to control the rate of rotation of the shaft and thus the speed in which the third blade member oscillates.

It is expected that equivalent drive means may also be utilized to impart such motion to the blades of the cast removal device, including but not limited to reciprocating devices that directly impart up and down motion to the blade member, back and forth reciprocating devices that, along with an offset pivot point would impart up and down motion to the blade member. Instead of electric motors, it is also contemplated that pneumatic motors or manually driven devices may be utilized to accomplish the same goals and results.

In operation, the motor 91 is actuated by pressing of the trigger 102, which imparts rotary motion to the shaft 92 via transmission 93. The rotary motion of the shaft 92 imparts an eccentric motion to the race assembly due to the offset nature of the opening there through. The outer surface first race member 96 engages the cam surfaces 58 of the second end of the third blade member and the eccentric motion of the race causes the second end of the third blade member to oscillate as the third blade member pivots about its pivot point. The leading end of the third blade member likewise oscillates up and down creating a double scissor-like action with respect to the first and second blade members.

To remove a cast, the operator positions the third blade member between the inner stocking of the cast and the patient's skin and positions the first and second blade members on the outer surface of the cast. The motor is then activated causing the third blade member to move up and down, thereby causing the first and second cutting edges thereof to engage and cut through the stocking and padding material. This motion also causes the cutting edges of the first and second blade members to engage and cut through the outer hard surface of the cast. This double-scissor cutting action causes a narrow strip of the cast to be cut away from the main body of the cast. The radius of curvature of the third blade member causes the removed cast material to be directed to the outside of the housing during operation to prevent jamming of the device.

The present invention, therefore, is well adapted to carry out the objects and attain the ends and advantages mentioned as well as others inherent therein. While presently preferred embodiments of the invention are given for the purpose of disclosure, numerous changes in the details will readily suggest themselves to those skilled in the art and which are encompassed within the spirit of the invention and the scope of the appended claims.

We claim:

1. A cast removal device comprising:
first and second blade members, each of said first and second blade members having a first planar surface and a second planar surface, a first longitudinal edge and a second longitudinal edge, first and second ends, and an opening disposed proximal to a midpoint along said first and second planar surfaces, said first and second blade members being positioned parallel to one another such that the second planar surface of the second blade member faces the second planar surface of the first blade member;
a third blade member having a first planar surface and a second planar surface, a first longitudinal edge and a second longitudinal edge, first and second ends, and a pivot point disposed proximal to a midpoint along said first and second planar surfaces for permitting oscillation about an axis perpendicular to said first and second planar surfaces; wherein said third blade member is positioned between said first and second blade members such that said first planar surface of said third blade member slidably engages said second surface of said first blade member and said second planar surface of said third blade member slidably engages said second planar surface of said second blade member;
means for rotatably connecting said first, second and third blade members at said pivot points thereof;
means for securing said first and second blade members together to prevent rotation with respect to one another; and
drive means for effecting oscillation of the third blade member in relation to the first and second blade members about said axis.

2. The cast removal device of claim 1, further comprising a V-shaped groove along a portion of the length of the first longitudinal edge of the third blade member such that a first cutting edge is formed at the intersection of the first planar surface and the first longitudinal edge and a second cutting edge is formed at the intersection of the second planar surface and the first longitudinal edge.

3. The cast removal device of claim 2, wherein the walls of the V-shaped groove are at an angle of between 20° and 45° in relation to a plane parallel to the first longitudinal edge.

4. The cast removal device of claim 3, wherein the walls of the V-shaped groove are at an angle of 45° in relation to a plane parallel to the first longitudinal edge.

5. The cast removal device of claim 2, wherein the portion of the length of the first longitudinal edge carrying said first and second cutting edges is curved.

6. The cast removal device of claim 5, wherein the radius of curvature is between 3″ and 3.5″.

7. The cast removal device of claim 6, wherein the radius of curvature is 3.2″.

8. The cast removal device of claim 2, further comprising a beveled section along a portion of the length of the first longitudinal edge of each of the first and second blade members such that cutting edges are formed at the intersection of the second planar surface and the second longitudinal edge of the first blade member and at the intersection of the second planar surface and the second longitudinal edge of the second blade member.

9. The cast removal device of claim 8, wherein the beveled surfaces are at an angle of between 10° and 20° in relation to a plane parallel to the second longitudinal edge.

10. The cast removal device of claim 9, wherein the beveled surfaces are at an angle of 15° in relation to a plane parallel to the second longitudinal edge.

11. The cast removal device of claim 1, wherein the first ends of said first, second and third blade members are rounded.

12. The cast removal device of claim 11, further comprising a cover removably disposed over the first end of said third blade member.

13. The cast removal device of claim 11, wherein the cover is formed of plastic material.

14. The cast removal device of claim 11, wherein the cover further comprises a projection proximal to a distal end thereof and a corresponding notch is provide in the second longitudinal edge of the third blade member for receiving said projection therein.

15. The cast removal device of claim 1, further comprising a blade housing wherein said first and second blade members are fixedly secured to said housing.

16. The cast removal device of claim 15, wherein said blade housing is formed of aluminum.

17. The cast removal device of claim 15, wherein said drive means comprises:
an electric drive motor for imparting rotary motion to a shaft passing through windings of said motor;
a first cylindrical race member having an opening offset from the center thereof for receiving a first end of said shaft;
a second cylindrical race member surrounding said first cylindrical race member;
a plurality of needle bearings disposed between the inner surface of said second cylindrical race member and the outer surface of said first cylindrical race member; and
a nut secured to a threaded end of said shaft for securing said first and second race members in place on said shaft.

18. The cast removal device of claim 17, wherein said second end of said third blade member comprises a semicircular opening therein having cam surfaces for selectively engaging and disengaging the outer surface of the second race member during rotation thereof.

19. The cast removal device of claim 1, wherein said blade members are formed from stainless steel.

20. The cast removal device of claim 1, wherein said blade members are formed from steel coated with a ceramic-metal matrix material.
